(19) United States
(12) Patent Application Publication
(10) Pub. No.: US 2015/0190563 A1
(43) Pub. Date: Jul. 9, 2015

(54) HYDROPHILIC RESIN COMPOUND HAVING SUGAR CHAIN AFFIXED THERETO, POLYMER SUBSTRATE FOR VIRUS-REMOVAL, AND BIOCOMPATIBLE MATERIAL

(71) Applicants: DIC Corporation, Tokyo (JP); National University Corporation Hamamatsu University School of Medicine, Hamamatsu-shi (JP)

(72) Inventors: Hirohide Nakaguma, Sakura-shi (JP); Naoto Sakurai, Sakura-shi (JP); Naoya Ikushima, Sakura-shi (JP); Tetsuro Suzuki, Hamamatsu-shi (JP)

(21) Appl. No.: 14/415,379

(22) PCT Filed: Jul. 19, 2013
(86) PCT No.: PCT/JP2013/069683

§ 371 Date: Jan. 16, 2015

(30) Foreign Application Priority Data
Jul. 20, 2012 (JP) 2012-161537
Apr. 10, 2013 (JP) 2013-082065

(51) Int. Cl. A61M 1/34; B01D 69/02; B01D 71/82; C08F 216/06; B01D 69/08

(52) U.S. Cl. CPC A61M 1/34; C08F 216/06; B01D 69/08; B01D 71/82; B01D 69/02; A61M 2202/206; A61M 2202/0413; B01D 2323/36

(57) ABSTRACT

Provided is: a resin compound having an immobilized sugar chain, obtained by reacting a an epoxy-group-containing compound (B) with a hydrophilic resin (A), followed by reacting an amino-group-containing compound (C) therewith, and then reacting a sugar therewith; a virus-removal-polymer substrate obtained by coating the resin compound on a polymer support to immobilize a sugar chain that can adsorb a virus; and a biocompatible material using the resin compound.

### TECHNICAL FIELD

The present invention relates to a hydrophilic resin compound having an immobilized sugar chain, a virus-removal-polymer substrate, a virus-removal apparatus, a method for operating the virus-removal apparatus, and a biocompatible material using the resin compound.

### BACKGROUND ART

As a hydrophilic resin compound having an immobilized sugar chain, a sugar-chain-bonded resin obtained by immobilizing a sugar chain onto a methacrylate-based polymer having an epoxy group (Patent Document 1) and a resin obtained by immobilizing a sugar chain onto an aminated vinyl chloride resin (Patent Document 2) have been disclosed. However, there are problems in that the resins lack an affinity for blood, because the main chain thereof is a methacrylate-based polymer or a vinyl chloride resin. In addition, an ion-binding-polymer-containing substrate having an ion-binding group and a sugar chain (Patent Document 3) has been disclosed. However, there is a problem in that the ion-binding polymer is adsorbed by a substrate due to the ion-binding properties thereof, and therefore is not applicable to a hydrophobic substrate material.

On the other hand, hepatitis C is caused by chronic hepatitis C virus (HCV) infection, and the general method for treating hepatitis C using medicine is a combination therapy of pegylated interferon and ribavirin. For patients in which the virus has the genotype 1 b and the viral load in the blood is high, the recovery ratio is about 50% and the likelihood of progression to hepatic cirrhosis or liver cancer is high, and therefore there has been a demand for the development of a more effective treatment and medicine (Non-Patent Document 1). In general, it is known that a treatment with medicine results in a high recovery ratio in the case of low viral load in the blood. It has been reported that, when removal of HCV in the blood through a porous filter is combined with a therapy using medicine, the recovery ratio is increased (Non-Patent Document 2). That is, the decrease in the viral load in the body probably resulted in an increase in the recovery ratio.

Patent Document 3 describes a blood processing apparatus in which a blood inlet, an upstream side blood channel, a plasma separation unit, and a downstream side blood channel are connected in this order; the plasma exit of the plasma separation unit, an upstream side plasma channel, a plasma purifying unit, and a downstream side plasma channel are connected in this order further; and the end of the downstream side plasma channel is connected to a blood-plasma mixing unit provided at an intermediate portion of the downstream side blood channel, wherein at least a blood cell processing unit including a water insoluble carrier for removing a virus and virs-infected cells is provided downstream of the blood-plasma mixing unit of the downstream side blood channel, and the plasma purifying unit is composed of a porous filter membrane having a maximum pore diameter of 20 nm or more and 50 nm or less.

However, the above-mentioned method employing removal with the filter is performed by temporarily achieving separation of blood cells and plasma and then removing a virus from the plasma component; and hence the channel configuration is complicated, and therefore there has been a demand for a simpler method of removing a virus from the blood.

As a blood-purification absorbent material for hepatitis C virus in which a ligand or the like is immobilized, Patent Document 4 describes a method in which a peptide having an affinity for immunoglobulin or the like is immobilized on a water-insoluble gel to efficiently remove immune-complex hepatitis C virus.

On the other hand, it is known that heparin is an effective ligand that can bind with HCV (Non-Patent Document 3). Accordingly, HCV may be removed more easily by using a substrate in which heparin is immobilized on a polymer support such as a hollow fiber through which whole blood can be passed without requiring separation of blood cells and plasma, or by using a substrate in which heparin is immobilized in, for example, pores of blood cell-plasma separation membrane, and thus it is expected that, for example, an HCV-removal module that puts a smaller load on patients can be provided.

The substrate on which heparin is immobilized may be in the form of a bead or a porous hollow fiber. Compared with extracorporeal circulation modules filled with susbtrates having particulate heparin immobilized thereon, internal-circulation or filtration-type extracorporeal circulation modules using porous hollow fibers have fewer portions where the blood stangnates and hence are advantageous in that the configuration is less likely to cause formation of blood clots. In the case of immobilizing heparin on a porous hollow fiber, the type of surface functional group and the immobilization density vary depending on the material of the substrate, and therefore an optimum method needs to be found in accordance with the substrate.

On the other hand, as a virus absorbent having a sugar, a substrate that can sorb human immunodeficiency virus (hereinafter, referred to as HIV) has been reported. For example, Patent Document 5 describes an HIV-adsorption polymer substrate having a sugar chain and obtained in the following manner: a polymerizable compound having an ethylenically unsaturated bond and a sugar chain or a polymerizable composition having the polymerizable compound is brought into contact with a polymer substrate having methylene groups as the main chain thereof and then irradiated with ionizing radiation; or the polymer substrate is irradiated with ionizing radiation and subsequently the polymerizable compound or a polymerizable composition having the polymerizable compound is brought into contact therewith.

### DOCUMENTS OF RELATED ART

### Patent Documents

Patent Document 1: Japanese Unexamined Patent Application, First Publication No. Sho 64-63038
Patent Document 2: Japanese Unexamined Patent Application, First Publication No. Hei 9-108331
Patent Document 3: Japanese Laid-Open Patent Application No. 2004-346209
Patent Document 4: Japanese Laid-Open Patent Application No. 2004-230165
Patent Document 5: Japanese Unexamined Patent Application, First Publication No. Hei 10-323387
Patent Document 6: Japanese Laid-Open Patent Application No. 2010-68910

### Non-Patent Documents

Non-Patent Document 1: Viral Hepatitis: Advances in Basic and Clinical Research, Nippon Rinsho, vol. 69, Special Issue vol. 4 (2011)
Non-Patent Document 2: A. K. Fujiwara et al., Heptatol. Res., 37, 701 (2007)
Non-Patent Document 3: Zahn, J. P. Allain, J. Gen. Virol., 86, 677 (2005)

### SUMMARY OF THE INVENTION

### Problems to be Solved by the Invention

In view of the related art, an object of the present invention is to provide: a hydrophilic resin having an immobilized sugar chain which has a high affinity for blood and makes it possible to realize immobilization onto a non-ionic base material; and a substrate and an apparatus, through which a body fluid such as blood can be passed without causing clogging due to formation of a water-resistant blood clot or the like, to thereby allow efficient removal of a virus in the body fluid.

In addition, the present invention aims to provide a biocompatible material using the resin compound.

### Means to Solve the Problems

In order to achieve the above-mentioned problems, the inventors of the present invention obtained a resin compound having an immobilized sugar chain by reacting a hydrophilic resin (A) with an epoxy-group-containing compound (B), followed by reacting an amino-group-containing compound (C) therewith, and then reacting a sugar with the resultant. In addition, the present inventors found that the above-mentioned problems can be solved by applying the resultant resin on a polymer support to immobilize a sugar chain that can adsorb a virus.

That is, the present invention relates to a resin compound obtained by reacting a hydrophilic resin (A) selected from the group consisting of ethylene-vinyl alcohol copolymers, ethylene-acrylic acid copolymers, and ethylene-vinyl alcohol-vinyl acetate copolymers, with an epoxy-group-containing compound (B), followed by reacting an amino-group-containing compound (C) therewith, and then reacting an amino group thereof with a sugar.

In addition, the present invention relates to a virus-removal-polymer substrate characterized by containing a surface coated with the resin compound.

In addition, the present invention relates to a virus-removal apparatus using the virus-removal-polymer substrate.

In addition, the present invention relates to a method for operating a virus-removal-apparatus, containing a step in which a fluid which has passed through pores of a porous hollow fiber and a fluid which has not passed through the pores thereof are mixed by passing a fluid containing a virus through the porous hollow fiber.

In addition, the present invention relates to a biocompatible material using the resin compound.

### Effects of the Invention

The present invention can provide: a resin compound that has an affinity for blood and is also applicable to a hydrophobic substrate; a polymer substrate that can selectively remove a virus without adsorbing or removing blood components that should not be removed; and a medical appliance using the same.

In addition, the present invention can provide a biocompatible material to be used for medical purpose by using the resin compound according to the present invention to prepare the biocompatible material.

### BRIEF DESCRIPTION OF THE DRAWINGS

FIG. 1 is a schematic cross-sectional view that indicates an aspect of a medical appliance including a polymer substrate according to the present invention.

### EMBODIMENTS FOR CARRYING OUT THE INVENTION

That is, the present invention relates to:

(1) a resin compound obtained by reacting a hydrophilic resin (A) selected from the group consisting of ethylene-vinyl alcohol copolymers, ethylene-acrylic acid copolymers, and ethylene-vinyl alcohol-vinyl acetate copolymers, with an epoxy-group-containing compound (B), followed by reacting an amino-group-containing compound (C) therewith, and then reacting a sugar with an amino group thereof;
(2) the resin compound according to (1) described above, wherein the epoxy-group-containing compound (B) is an epichlorohydrin or a diepoxy compound;
(3) the resin compound according to (1) or (2) described above, wherein the amino-group-containing compound (C) is an ammonia, a methylamine, an ethylamine, a 2-aminoethanol, an ethylenediamine, a butylenediamine, a hexamethylenediamine, a 1,2-bis(2-aminoethoxy) ethane, a 3,3′-diaminodipropylamine, a diethylenetriamine, a phenylenediamine, a polyallylamine, or a polyethyleneimine;
(4) the resin compound according to any one of (1) to (3) described above, wherein the sugar is a heparin, a heparin derivative obtained by subjecting a primary or secondary hydroxyl group of heparin to sulfuric-esterification, a heparin derivative obtained by removing an N-acetyl group from heparin to obtain a deacetylated heparin, and then subjecting the deacetylated heparin to N-sulfuric-esterification, a heparin derivative obtained by removing an N-sulfate group from heparin to obtain a desulfated heparin, and then subjecting the desulfated heparin to N-acetylation, a low-molecular-weight heparin, a dextran sulfate, a fucoidan, a chondroitin sulfate A, a chondroitin sulfate C, a dermatan sulfate, a heparinoid, a heparan sulfate, a rhamnan sulfate, a ketaran sulfate, an alginic acid, a hyaluronic acid, or a carboxymethyl cellulose;
(5) the resin compound according to any one of (1) to (4) described above, wherein the hydrophilic resin (A) is an ethylene-vinyl alcohol copolymer or an ethylene-vinyl alcohol-vinyl acetate copolymer, in which a molar ratio of ethylene to vinyl alcohol, ethylene/vinyl alcohol, is within a range of 0.5 to 1.0;
(6) a virus-removal-polymer substrate, containing a surface coated with the resin compound of any one of (1) to (5) described above;
(7) the virus-removal-polymer substrate according to (6) described above, wherein a virus is a hepatitis virus;
(8) the virus-removal-polymer substrate according to (6) or (7) described above, wherein a polymer substrate is a porous hollow fiber, a non-woven fabric, or a dialysis membrane;
(9) the virus-removal-polymer substrate according to (8) described above, wherein the polymer substrate is a porous hollow fiber;
   (10) the virus-removal-polymer substrate according to (9) described above, wherein the porous hollow fiber has an mean flow pore size within a range of 50 to 500 nm;
   (11) the virus-removal-polymer substrate according to (9) or (10) described above, wherein the porous hollow fiber has an inner diameter within a range of 150 to 500 μm;
   (12) the virus-removal-polymer substrate according to any one of (9) to (11) described above, wherein the porous hollow fiber has a membrane thickness within a range of 30 to 100 μm;
   (13) a virus-removal-apparatus using the virus-removal-polymer substrate of any one of (6) to (8) described above;
   (14) a virus-removal-apparatus using the virus-removal-polymer substrate of any one of (9) to (12) described above;
   (15) a method for operating a virus-removal-apparatus of (14) described above, containing a step in which a fluid which has passed through pores of a porous hollow fiber and a fluid which has not passed through the pores thereof are mixed by passing a fluid containing a virus through the porous hollow fiber;
   (16) a method for operating a virus-removal-apparatus according to (15) described above, wherein the fluid containing a virus is a blood containing a virus; and
   (17) a biocompatible material using the resin compound of any one of (1) to (5) described above.

In the following, the present invention will be explained in detail.

A resin compound according to the present invention is obtained by reacting a hydrophilic resin (A) with an epoxy-group-containing compound (B), followed by reacting an amino-group-containing compound (C) therewith, and then reacting an amino group thereof and a sugar.

Hydrophilic Resin (A)

A hydrophilic resin (A) available in the present invention is selected from the group consisting of ethylene-vinyl alcohol copolymers, ethylene-acrylic acid copolymers, and ethylene-vinyl alcohol-vinyl acetate copolymers. Among these, an ethylene-vinyl alcohol copolymer or an ethylene-vinyl alcohol-vinyl acetate copolymer is preferable. Since the resin compound has a hydroxyl group, the resin compound has a high affinity for blood, which is preferable. In the case where the ethylene-vinyl alcohol copolymer or the ethylene-vinyl alcohol-vinyl acetate copolymer is used, the molar ratio of ethylene to vinyl alcohol is preferably within a range of 0.5 to 1.0. In the case where the molar ratio of ethylene to vinyl alcohol is 0.5 or more, the water-resistant of the resin is improved. In the case where the molar ratio is 1.0 or less, the hydrophilicity of the resin is improved, and the surface hydrophilization effects of the resin compound having an immobilized sugar chain (resin for surface treatment) are improved, which are preferable.

As the molecular weight distribution of the hydrophilic resin (A), the weight-mean molecular weight thereof is preferably 10000 to 300000. In the case where the weight-mean molecular weight is 10000 or more, the water-resistant of the resin is improved. In the case where the weight-mean molecular weight is 300000 or less, the solubility to a solvent is improved. In the present specification, the weight-mean molecular weight denotes the weight-mean molecular weight measured by gel permeation chromatography (GPC) compared to standard polystyrene.

Epoxy-Group-Containing Compound (B)

An epoxy-group-containing compound (B) available in the present invention is used to form a bridge between the hydrophilic resin (A) described above and an amino-group-containing compound (C) described below. Accordingly, it is required to have a functional group that can react with an amino group after reaction with the hydrophilic compound (A). Examples thereof include an epichlorohydrin, diepoxy compounds, and polyepoxy compounds. Among these, the epichlorohydrin or the diepoxy compounds are preferably used, and the epichlorohydrin is more preferably used.

Reaction between hydrophilic resin (A) and epoxy-group-containing compound (B)

The hydrophilic resin (A) and the compound (B) may be reacted using conventionally-known various methods. Among the methods, it is preferable that the hydrophilic resin (A) and the compound (B) be uniformly reacted in a solvent in which both the hydrophilic resin (A) and the compound (B) can be dissolved. Examples of the solvent include: aprotic polar solvents, such as a dimethyl sulfoxide and a dimethylformamide; solvent mixtures composed of an alcohol and water, such as those composed of an ethanol and water; an n-propanol and water; a methanol and water; or, an isopropyl alcohol and water; a pyridine, a phenol, a cresol, and the like. The solvents may be used alone or in combination thereof. The reaction may be performed at 40 to 100° C. for 10 minutes to 20 hours to obtain the reaction product.

In the case where an ethylene-vinyl alcohol copolymer or an ethylene-vinyl alcohol-vinyl acetate copolymer is used as the hydrophilic resin (A), dimethyl sulfoxide is preferably used as a solvent in which the hydrophilic resin (A) and the epoxy-group-containing compound (B) are to be reacted, because the solubilities thereof are high and the side reaction is suppressed. In addition, in the case where an ethylene-vinyl alcohol copolymer or an ethylene-vinyl alcohol-vinyl acetate copolymer is used, it is preferable that a base catalyst such as a sodium hydroxide or a potassium hydroxide be added thereto to promote the reaction, and the preferable addition amount thereof is within a range of 0.38 to 3.8 mmol, more preferably 0.75 to 2.0 mmol, with relative to 1 g of the hydrophilic resin (A). It is preferable that the reaction product obtained by reacting the hydrophilic resin (A) and the epoxy-group-containing compound (B) have an epoxy equivalent of 370 to 3700 g/mol, more preferably 530 to 2775 g/mol, and even more preferably 690 to 1850 g/mol.

Amino-Group-Containing Compound (C)

An amino-group-containing compound (C) is used in the present invention to introduce an amino group into the reaction product of the hydrophilic resin (A) and the epoxy-group-containing compound (B). Examples thereof include an ammonia, a methylamine, an ethylamine, a 2-aminoethanol, an ethylenediamine, a butylenediamine, a hexamethylenediamine, a 1,2-bis(2-aminoethoxy) ethane, a 3,3′-diaminodipropylamine, a diethylenetriamine, a phenylenediamine, a polyallylamine, a polyethyleneimine, and the like. Among these, an ammonia, a methylamine, an ethylamine, a 2-aminoethanol, and others that hardly cause gelation are preferable, because polyvalent amino compounds easily cause gelation of the resin.

Reaction between: reaction product of hydrophilic resin (A) and epoxy-group-containing compound (B); and amino-group-containing compound (C)

The reaction product of the hydrophilic resin (A) and the compound (B) may be reacted with an amino-group-containing compound (C) using conventionally-known various methods. Among the methods, it is preferable that the reaction product of the hydrophilic resin (A) and the compound (B) be uniformly reacted with an amino-group-containing compound (C) in a solvent in which both the reaction product and the amino-group-containing compound (C) can be dissolved. Examples of the solvent include: aprotic polar solvents, such as a dimethyl sulfoxide and a dimethylformamide; solvent mixtures composed of an alcohol and water, such as those composed of: an ethanol and water; an n-propanol and water; a methanol and water; or, an isopropyl alcohol and water; a pyridine, a phenol, a cresol, and the like. The solvents may be used alone or in combination. Among these, a solvent mixture composed of an alcohol and water is preferably used, in terms that the boiling point thereof is low, which allows easy drying after coating. The reaction may be performed at 40 to 100° C. for 10 minutes to 20 hours to obtain the reaction product. It is preferable that the amount of an amino group to be introduced in the resin for surface treatment be an amine number of 15 to 150 mg KOH/g, and more preferably 30 to 80 mg KOH/g.

Sugar

Although various conventionally-known sugars may be used in the present invention, a sugar that can efficiently capture a virus using action such as adsorbent action to remove the virus from a fluid containing the virus is preferably used. Examples thereof include: heparin; heparin derivatives obtained by subjecting a primary or secondary hydroxyl group of heparin to sulfuric-esterification; heparin derivatives obtained by removing an N-acetyl group from heparin to obtain a deacetylated heparin, and then subjecting the deacetylated heparin to N-sulfuric-esterification; heparin derivatives obtained by removing an N-sulfate group from heparin to obtain a desulfated heparin, and then subjecting the desulfated heparin to N-acetylation; a low-molecular-weight heparin, a dextran sulfate, a fucoidan, a chondroitin sulfate A, a chondroitin sulfate C, a dermatan sulfate, a heparinoid, a heparan sulfate, a rhamnan sulfate, a ketaran sulfate, an alginic acid, a hyaluronic acid, and a carboxymethyl cellulose.

As heparin, conventionally-known heparin may be used without limitation. Heparin is widely distributed in the body such as the small intestine, the muscle, the lungs, the spleen, and mast cells. Chemically, heparin is a kind of heparan sulfate, which is a glycosaminoglycan. Heparin is a polymer in which β-D-glucuronic acid or α-L-iduronic acid is polymerized with D-glucosamine through 1,4-bonds. Heparin has a feature of having a very high degree of sulfation, compared with heparan sulfate.

Although the weight-mean molecular weight of heparin is also not particularly limited, heparin having a high weight-mean molecular weight has low reactivity with the compound (C) and hence the immobilization efficiency of heparin is probably low. Accordingly, the weight-mean molecular weight of heparin is preferably approximately 500 to 500,000 daltons, more preferably 1,200 to 50,000 daltons, and still more preferably 5,000 to 30,000 daltons.

A heparin derivative available in the present invention is preferably a heparin derivative obtained by subjecting a primary or secondary hydroxyl group of heparin to sulfuric-esterification, a heparin derivative obtained by removing an N-acetyl group from heparin to obtain a deacetylated heparin, and then subjecting the deacetylated heparin to N-sulfuric-esterification, or a heparin derivative obtained by removing an N-sulfate group from heparin to obtain a desulfated heparin, and then subjecting the desulfated heparin to N-acetylation.

In the case of synthesizing the heparin derivative obtained by subjecting a primary or secondary hydroxyl group of heparin to sulfuric-esterification, for example, an alkali salt of the heparin is passed through an ion-exchange resin (H+) or the like and treated with an amine to prepare a heparin amine salt. Thereafter, the heparin amine salt is treated with a sulfating agent to obtain the target heparin derivative. The sulfating agent is preferably conventionally-known SO₃-pyridine or the like.

In the case of synthesizing the heparin derivative obtained by removing an N-acetyl group from heparin to obtain a deacetylated heparin, and then subjecting the deacetylated heparin to N-sulfuric-esterification, for example, an N-acetyl group of heparin is deacetylated with hydrazine or the like, and then the resultant is treated with a sulfating agent to obtain the target heparin derivative. The sulfating agent is preferably conventionally-known SO₃—NMe₃ or the like.

In the case of synthesizing the heparin derivative obtained by removing an N-sulfate group from heparin to obtain a desulfated heparin, and then subjecting the desulfated heparin to N-acetylation, for example, a pyridinium salt of heparin is prepared, and then only sulfate groups on nitrogen atoms are desulfated, followed by performing N-acetylation using a conventionally-known method.

As the low-molecular-weight heparin, the dextran sulfate (having a sulfur content of 3 to 6% by weight), the dextran sulfate (having a sulfur content of 15 to 20% by weight), the fucoidan, the chondroitin sulfate A, the chondroitin sulfate C, the dermatan sulfate, the heparinoid, the heparan sulfate, the rhamnan sulfate, the ketaran sulfate, the alginic acid, the hyaluronic acid, and the carboxymethyl cellulose, conventionally-known ones are available.

The sulfation degree of the dextran sulfate may be high (the sulfur content thereof is 15 to 20% by weight) or low (the sulfur content thereof is 3 to 6% by weight), and there is no particular limitation on the sulfation degree, provided that the dextran sulfate can be obtained using a conventionally-known method.

Heparinoid denotes sulfated polysaccharides that are generally described in “The Japanese pharmaceutical codex” and the like. However, the heparinoid is not limited to those described in “The Japanese pharmaceutical codex”, provided that the heparinoid can be obtained using a conventionally-known extraction method or preparation method.

Among the sugars, heparin and heparinoid are preferable, in terms that the virus-adsorbability thereof is high.

Immobilization of a sugar via the amino-group-containing compound (C) requires that the compound (C) and the sugar are bonded by a covalent bond. Such a bond may be formed by appropriately performing a conventionally-known reaction.

The reaction to immobilize the sugar is preferably an amidation reaction or a reduction amination reaction. As the amidation method, for example, a conventionally-known amidation reaction used to synthesize peptide or the like, such as, amidation with an active ester, amidation with a condensing agent, the combination thereof, a mixed acid anhydride method, an azide method, an oxidation-reduction method, a DPPA method, or a Woodward method may be appropriately performed. The reduction amination reaction may be performed using a conventionally-known method in which the reaction between an amino group of the compound (C) and the reducing terminal of the sugar is caused.

Amidation with an active ester may be performed, for example, by the following method: an active ester in which a highly cleavable group is temporarily condensed with a carboxy group is formed using an NHS (N-hydroxysuccinimide), a nitropheno, a pentafluorophenol, a DMAP (4-dimethylaminopyridine), a HOBT (1-hydroxybenzotriazole), a HOAT (hydroxyazabenzotriazole), or the like, and then reacted with an amino group. Although amidation with a condensing agent may be performed alone, the amidation may be performed in combination with the active ester. Examples of the condensing agent include EDC (1-(3-dimethylaminopropyl-3-ethyl-carbodiimidehydrochloride), HONB (endo-N-hydroxy-5-norbornene-2,3-dicarboxamide), DCC (dicyclohexylcarbodiimide), BOP (benzotriazole-1-yloxytris(dimethylamino)phosphonium hexafluorophosphate), HBTU (O-benzotriazole-1-yl-N,N,N′,N′-tetramethyluronium hexafluorophosphate), TBTU (O-benzotriazole-1-yl-N,N,N′,N′-tetramethyluronium tetrafluoroborate), HOBt (1-hydroxybenzotriazole), HOOBt (3,4-dihydro-3-hydroxy-4-oxo-1,2,3-benzotriazine), di-p-trioylcarbodiimide, DIC (diisopropylcarbodiimide), BDP (1-benzotriazolediethylphosphate-1-cyclohexyl-3-(2-morpholinylethyl) carbodiimide), cyanuric fluoride, cyanuric chloride, TFFH (tetramethylfluorformamidinium hexafluorophosphae), DPPA (diphenylphosphorazidate), TSTU (O—(N-succinimidyl)-N,N,N′,N′-tetramethyluronium tetrafluoroborate), HATU (N-[(dimethylamino)-1-H-1,2,3-triazolo[4,5,6]-pyridine-1-ylmethylene]-N-methylmethaneaminium hexafluorophosphate N-oxide), BOP-Cl (bis(2-oxo-3-oxazolidinyl)phosphine chloride), PyBOP ((1-H-1,2,3-benzotriazole-1-yloxykris(pyrrolidino) phosphonium tetrafluorophosphate), BrOP (bromotris(dimethylamino) phosphonium hexafluorophosphate), DEPBT (3-(diethoxyphosphoryloxy)-1,2,3-benzotriazin-4(3H)-one), and PyBrOP (bromotris(pyrrolidino) phosphonium hexafluorophosphate).

As a solvent available in the amidation method, water or an organic solvent available in peptide synthesis may be used, and examples thereof include a dimethylformamide (DMF), a dimethyl sulfoxide (DMSO), a hexaphosphoroamide, a dioxane, a tetrahydrofuran (THF), an ethyl acetate, solvent mixtures composed of an alcohol and water, such as, those of: an ethanol and water; an n-propanol and water; a methanol and water; or an isopropyl alcohol and water, a pyridine, a phenol, a cresol, solvent mixtures thereof, and aqueous solutions containing the same.

Examples of a reductant available in the reduction amination reaction include reductants, such as a sodium borocyano trihydride, a sodium triacetoxyborohydride, a pyridine borane, a picoline borane, and the like.

The reaction may be conducted at 20 to 100° C. for 10 minutes to 100 hours, approximately, to obtain a target reaction product. It is more preferable that the reaction be conducted approximately at 20 to 60° C., since hydrolysis reaction of the sugar may progress at a high temperature, for example.

Amidation Reaction of Amino Group

In the case where an unreacted amino group remains in the resin compound having an immobilized sugar chain according to the present invention, the unreacted amino group probably interacts with a carboxyl group or a sulfate group in the sugar chain to form ion complex, and therefore effects of the resin compound are probably not be maximized. Accordingly, it is preferable that the remaining unreacted amino group be amidated.

The amidation reaction may be conducted using a conventionally-known method, and examples of the method include: a method in which an amino group is amidated by reacting the amino group with an acid anhydride, such as an acetic anhydride, a propionic anhydride, a butanoic anhydride, a hexanoic anhydride, an anhydrous citric acid, a phthalic anhydride, or a maleic anhydride; and a method in which a halogenated carboxylic acid compound, such as an acetyl chloride, a propionyl chloride, a butyryl chloride, or a hexanoyl chloride, is used. In addition, amidation may be conducted using a carboxylic acid with an active ester described with respect to a method to immobilize sugar, or a condensing agent.

Among these, it is preferable that amidation be conducted using a halogenated carboxylic acid compound, and more preferable that a basic compound, such as a trimethylamine, a triethylamine, or a pyridine, be added thereto so as to trap generated halogenated hydrogen to allow smooth progression of the reaction.

Such an amidation reaction may be conducted, for example, by dissolving the resin compound having an immobilized sugar chain in DMSO, adding acetyl chloride with the above-mentioned basic compound thereto at 0° C. to 40° C., and then reacting the mixture for 1 to 3 hours.

It is preferable that the amount of a sugar chain (D) in the resin compound having an immobilized sugar chain according to the present invention be 1 to 40% by weight, and more preferably 1 to 20% by weight, with respect to the total weight of the resin compound having an immobilized sugar chain. In the case where the amount is 1% by weight or more, the virus-removal efficiency is improved, while in the case where the amount is 40% by weight or less, the water-resistant of the resin is improved.

A virus-removal-polymer substrate according to the present invention is prepared using the above-mentioned resin compound. The virus-removal-polymer substrate according to the present invention preferably has a surface layer containing the resin compound. The surface layer is preferably formed by coating the resin compound on the surface of a polymer support.

Although the form of the virus-removal-polymer substrate is not particularly limited and can be selected from various forms such as a porous hollow fiber, a bead, a non-woven fabric, and a dialysis membrane, the form is preferably a porous hollow fiber, a non-woven fabric, or a dialysis membrane.

Various kinds of conventionally-known polymer substrate (polymer support) may be used in the present invention, examples thereof include olefin resins, styrene resins, sulfone resins, acrylic resins, urethane resins, ester resins, ether resins, and cellulose mixed esters, and specific examples thereof include high-density polyethylene, polyethylene terephthalate, polymethyl methacrylate, polysulfone, polyethersulfone, polyacrylonitrile, polyethylene, polypropylene, poly-4-methylpentene, triacetylcellulose, and regenerated cellulose.

The virus-removal-polymer substrate according to the present invention may be obtained by coating the resin compound having an immobilized sugar chain according to the present invention on the surface of the polymer substrate (polymer support). The available polymer support is not particularly limited, and can be selected from various forms such as a porous hollow fiber, a bead, a non-woven fabric, and a dialysis membrane.

The coating method may be selected from various conventionally-known methods. Preferable examples thereof include a method in which a polymer support is immersed in a solution of the resin compound having an immobilized sugar chain according to the present invention, followed by pulling out and then drying the polymer support. The increase in the ratio of the resin solid content of the resin compound having an immobilized sugar chain on the polymer support, relative to the amount of the solution required to immobilize the resin compound having an immobilized sugar chain on the polymer support (resin solid content (parts by weight)/solution amount (parts by volume)) increases the amount of the resin to be treated in a reaction vessel with the same capacity, and therefore the reaction efficiency is increased, which allows a decrease in the production cost.

Alternatively, the virus-removal-polymer substrate according to the present invention may be obtained by rendering porous a mixture of polyolefin or the like (polymer support) and the resin compound having an immobilized sugar chain according to the present invention.

Alternatively, the virus-removal-polymer substrate according to the present invention may be obtained by spinning or forming the resin compound having an immobilized sugar chain according to the present invention in the form of a porous hollow fiber, a bead, a non-woven fabric, or the like, using various conventionally-known methods.

The amount of the sugar immobilized in the virus-removal-polymer substrate is not particularly limited, provided that a virus can be efficiently removed. However, in the case of extracorporeal circulation, biocompatibility is important, and therefore it is necessary to adjust the amount so as to prevent occurrence of adsorption of plasma proteins or activation of complements. In such a case, the amount of the immobilized sugar can be adjusted by controlling the amount of the amino-group-containing compound (C) to be introduced, or by modifying reaction conditions for immobilizing sugar, for example. Studies have revealed that the preferable amount of the immobilized sugar is 1 to 100 μg/cm², more preferably 2 to 80 μg/cm², and even more preferably 3 to 70 μg/cm².

In the case where the virus-removal-polymer substrate according to the present invention is a porous hollow fiber, the porous hollow fiber may be prepared using a conventionally-known method, depending on the intended usage purpose. In the case of a polyolefin porous hollow fiber, ones having various fine pore size and pore size distribution may be prepared by subjecting a spun fiber to an annealing treatment, cold drawing, hot drawing, and heat fixing.

In the case where the virus-removal-polymer substrate according to the present invention is a porous hollow fiber, a virus can be efficiently removed by passing a fluid containing a virus through pores of the porous hollow fiber. In the case where the blood is treated during extracorporeal circulation, although the treatment of the whole blood through pores is simple and therefore desirable, it is more desirable that blood cells and a plasma component be separated from each other, and only the plasma component be passed through the pores to remove a virus from the plasma, in view of stagnation and the requirement of high biocompatibility, because of the direct contact of blood cells with pores. In such a case, a fluid which has passed through pores of a porous hollow fiber and a fluid which has not passed through the pores thereof are generated. Studies on the removal ratio of the virus in the fluid containing a virus have revealed that, the removal ratio of the virus in the fluid which has passed through the pores of the porous hollow fiber is high and albumin, which is a useful component in the blood, is not removed therefrom. In addition, the removal ratio of the virus in the fluid which has passed through pores of the porous hollow fiber is higher than the fluid which has not passed through the pores of the porous hollow fiber, that is, the fluid which has come into contact with only the surface of the pores or pores in the region close to the surface, and it has been indicated that the viral-removal mainly occurs when the fluid passes through the pores of the porous hollow fiber.

Here, the term “which have passed through pores” denotes the state in which the fluid has passed from the inner surface to the outer surface of a porous hollow fiber or from the outer surfaces to the inner surfaces thereof.

It is not necessary for the pores of the porous hollow fiber to extend through the membrane as a straight tube, and may be bent within the membrane. Some pores may be integrated within the membrane, a single pore may be branched, or such structures may be simultaneously present.

In the case where the virus-removal-polymer substrate according to the present invention is a porous hollow fiber, the pore size of the porous hollow fiber is not particularly limited, provided that the pore size makes it possible to remove the virus efficiently. For example, in the case where efficient removal of the virus from plasma in extracorporeal circulation is aimed, the design described below is preferable. It is preferable, from the standpoint of the function of a plasma separation membrane required in the case where blood cells and plasma are separated from each other to remove virus from the plasma, that the mean flow pore size be 500 nm or less so as to prevent entry of blood cell components and blood platelets into cores. Furthermore, it is preferable that the mean flow pore size be 50 nm or more, so that the permeability of protein components in the plasma is not decreased. It is more preferable that the mean flow pore size be 50 to 500 nm so as to provide the function of a plasma separation membrane. Among these, the fine pore size of the porous hollow fiber is appropriately determined depending of the size of the target virus. For example, in the case of hepatitis C virus, the fine pore size (mean flow pore size) is preferably 80 to 250 nm, and more preferably 100 to 180 nm. Alternatively, in the case of a relatively large virus, such as human immunodeficiency virus, the fine pore size (mean flow pore size) is preferably 100 to 250 nm, and more preferably 120 to 200 nm.

In the case where the virus-removal-polymer substrate according to the present invention is a porous hollow fiber, the inner diameter of the porous hollow fiber is not particularly limited, provided that the inner diameter allows efficient removal of the virus. For example, in the case where the porous hollow fiber is used in extracorporeal circulation, it is preferable that the inner diameter of the porous hollow fiber be designed, as follows.

Since the amount of the blood that can be taken from the human body for circulation is limited, the size of the circulation module or the like cannot be excessively increased. In the case where the inner diameter is excessively large, the number of fibers that can be installed in the module is decreased, and thereby the contact area may be decreased or the linear velocity may become low to cause stagnation of the blood. On the other hand, in the case where the inner diameter is excessively small, the blood cell component probably tends to cause clogging. In consideration of the above-mentioned aspects, it is preferable that the inner diameter of the porous hollow fiber be 150 to 500 μm, more preferably 160 to 400 μm, and even more preferably 170 to 350 μm. Here, the inner diameter may be determined by conducting observation using an optical microscope or an electronic microscope.

In the case where the virus-removal-polymer substrate according to the present invention is a porous hollow fiber, the membrane thickness of the porous hollow fiber is not particularly limited, provided that efficient removal of the virus is allowed. For example, in the case where the virus is aimed to be efficiently removed from the plasma in extracorporeal circulation, it is preferable that the membrane thickness be 30 to 100 μm, more preferably 35 to 80 μm, and even more preferably 40 to 60 μm, in view of, for example, the plasma separation performance, the contact area, and the mechanical strength of the hollow fiber. Here, the membrane thickness is determined by conducting observation using an optical microscope or an electronic microscope.

In addition, the virus-removal-polymer substrate according to the present invention may have a constitution in which another substrate that can capture and remove a virus is combined in an outer portion of the porous hollow fiber. Such a constitution makes it possible to improve the removal ratio of the virus. Such another substrate is not particularly limited, provided that the substrate can capture and remove a virus, and examples thereof include a sugar-chain-immobilized gel and a sugar-chain-immobilized non-woven fabric.

In the case where a dialysis membrane is used as a polymer substrate, the resin compound according to the present invention may be coated on the surface of the dialysis membrane in the same manner as described above. The dialysis membrane to be used may be a conventionally-known one, and preferable examples of a material thereof include polysulfone, triacetyl cellulose and regenerated cellulose.

The dialysis membrane having a coated resin compound makes it possible to remove a virus in the blood while conducting dialysis, and therefore is particularly useful.

A method in which a sugar chain is immobilized onto a functional group on a substrate via a covalent binding has problems in which complicate processes are required, damage to a substrate may occur, and a large-scale washing process is required to prevent elution of reaction reagents or by-products. The method in which a resin compound having an immobilized sugar chain is coated on a substrate or a method in which a resin compound having an immobilized sugar chain is molded makes it possible to solve the problems, and thus it is believed that the surface treatment using a resin compound having an immobilized sugar chain is useful for providing medical apparatuses.

Fluid Containing a Virus

A target fluid containing a virus in the present invention is not particularly limited, provided that it is a fluid containing a virus. Specific examples thereof include a body fluid, which is a liquid component in the human body, and a culture fluid containing a virus. Specific examples of the body fluid include blood, saliva, perspiration, urine, snivel, semen, plasma, lymph, and tissue fluid.

The form of a medical appliance(virus-removal apparatus) including the virus-removal-polymer substrate according to the present invention is not particularly limited, provided that the form is usable in the above-mentioned applications, and examples thereof include a hollow fiber module, a filtration column, and a filter. In the case of a hollow fiber module or a filtration column, the form and material of a container thereof is not particularly limited. In the case of application to extracorporeal circulation of a body fluid (blood), a cylindrical container having an internal volume of 10 to 400 mL and an outer diameter of about 2 to 10 cm, more preferably a cylindrical container having an internal volume of 20 to 300 mL and an outer diameter of about 2.5 to 7 cm is preferable.

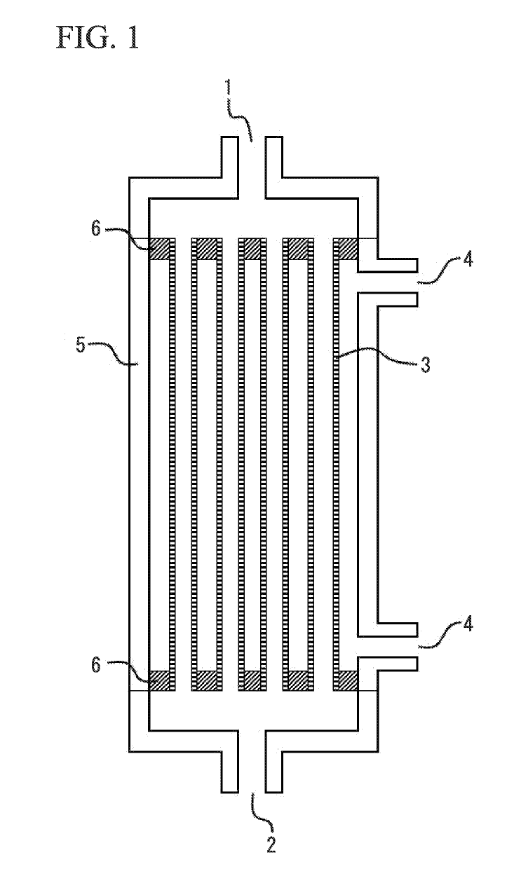

An embodiment of the virus-removal apparatus is shown in FIG. 1. In the virus-removal apparatus shown in FIG. 1, a virus-removal-polymer substrate (porous hollow fiber membrane) 3 is placed in a container 5. The adjacent porous hollow fiber membranes 3, 3 are arranged in parallel. Partitions 6 are placed between the porous hollow fiber membrane 3 and an internal wall of the container 5, and between the adjacent porous hollow fiber membranes 3, 3. A virus fluid inflow port (first opening part) 1 connecting to an internal space of the porous hollow fiber membrane 3 is formed in the middle of one end face in a longitudinal direction of the container 5. On the other hand, in the middle of the other end face of the container 5, an outlet of fluid which has not passed through pores (second opening part) 2 connecting to the virus fluid inflow port 1 via the internal space of the porous hollow fiber membrane 3 is formed. In addition, in an outer periphery of the container 5, an outlet of fluid which has passed through pores (third opening part) 4 connecting to the virus fluid inflow port 1 via the porous hollow fiber membrane 3 is formed.

In addition, although not shown in the drawing, it is preferable that the virus fluid inflow port 1, the outlet of fluid which has not passed through pores 2, and the outlet of fluid which has passed through pores 4 be configured to allow outflow fluids from the respective opening parts (outlets) to be mixed and then reintroduced into the virus-removal apparatus to be repeatedly subjected to a filtration process via the porous hollow fiber membrane 3, from the standpoint of improvement in the virus-removal efficiency.

In the case where a fluid containing a virus is introduced from the virus fluid inflow port 1 into the internal space of the porous hollow fiber membrane 3 in the virus-removal apparatus having such a configuration, the fluid passes from the inner surface of the porous hollow fiber membrane 3 to the outer surface side thereof, followed by mixing a fluid which has been exhausted from the external space of the porous hollow fiber membrane 3 to the outlet of fluid which has passed through pores 4 with a fluid which has come into contacting with the inner surface of the porous hollow fiber membrane 3 or pores in the region close to the inner surface and then has been exhausted from the internal space of the porous hollow fiber membrane 3 to the outlet of fluid which has not passed through pores 2, followed by reintroducing the mixture fluid into the virus-removal apparatus from the virus fluid inflow port 1.

On the other hand, in the case where the fluid containing a virus is introduced from one of the third opening parts 4, 4, into the external space of the porous hollow fiber membrane 3, the fluid passes from the outer surface of the porous hollow fiber membrane 3 to the inner surface side thereof, followed by mixing a fluid which has been exhausted from the internal space of the porous hollow fiber membrane 3 to the first opening part 1 or the second opening part 2 with a fluid which has come into contact with the outer surface of the porous hollow fiber membrane 3 or pores in the region close to the outer surface and then has been exhausted from the external space of the porous hollow fiber membrane 3 to the other of the third opening part 4, followed by reintroducing the mixture fluid into the virus-removal apparatus.

Although a method for operating (actuating) the virus-removal apparatus (medical appliance) according to the present invention may be any method that allows removal and separation of a virus in a fluid containing a virus by making the fluid contact therewith, a method for operating the virus-removal apparatus shown in FIG. 1 will be specifically explained below. First, the fluid containing a virus is introduced from the virus fluid inflow port 1. The introduced fluid containing a virus is directed to the porous hollow fiber membrane 3, and a virus is captured and removed by pores when the fluid containing the virus passes through the pores of the porous hollow fiber membrane 3. The fluid which has passed through pores of the porous hollow fiber membrane 3 is exhausted from the outlet of fluid which has passed through pores 4, and the fluid which has not passed through the pores of the porous hollow fiber membrane 3 is exhausted from the outlet of fluid which has not passed through pores 2.

In the case where the blood is used as the fluid containing a virus, it is preferable that the fluid which has been exhausted from the outlet of fluid which has not passed through pores 2 be mixed with the fluid which has been exhausted from the outlet of fluid which has passed through pores 4, the obtained mixture fluid be reintroduced from the virus fluid inflow port 1 into the porous hollow fiber membrane 3, and then the process for capturing and removing a virus by the pores of the porous hollow fiber membrane 3 be repeatedly conducted. The virus-removal efficiency can be further improved by repeatedly conducting the procedures.

In the case where the plasma is used as the fluid containing a virus, for example, the outlet of fluid which has not passed through pores 2 is closed, only a fluid which has been exhausted from the outlet of fluid which has passed through pores 4 to the outside of the apparatus is reintroduced from the virus fluid inflow port 1 to the porous hollow fiber membrane 3, and then the process for capturing and removing a virus at pores of the porous hollow fiber membrane 3 is repeatedly conducted.

Alternatively, the fluid containing a virus may be introduced from one of the third opening parts 4, 4 to the external space of the porous hollow fiber membrane 3, and then be allowed to pass through pores of the porous hollow fiber membrane 3 to capture and remove a virus at the pores. In such a case, the fluid which has passed from the outer surface of the porous hollow fiber membrane 3 to the inner surface side thereof is exhausted from the first opening part 1 or the second opening part 2, and a fluid which has come into contact with only the outer surface of the porous hollow fiber membrane 3 or the fine pores in the region close to the outer surface without passing from outer surface of the porous hollow fiber membrane 3 to the inner surface side thereof is exhausted from the other third opening part 4.

In the case where the blood is used as the fluid containing a virus, it is preferable that the fluid which has been exhausted from the first opening part 1 or the second opening part 2 be mixed with the fluid which has been exhausted from the third opening part 4, the mixture fluid be reintroduced from the third opening part 4 into the external space of the porous hollow fiber membrane 3, and then a process for capturing and removing a virus at pores of the porous hollow fiber membrane 3 be repeatedly conducted. The virus-removal efficiency can be improved by repeatedly conducting the procedure.

In the case where the plasma is used as the fluid containing a virus, it is also preferable that only fluids which has been exhausted from the first opening part 1 or the second opening part 2 be collected and then reintroduced from the third opening part 4 to the external space of the porous hollow fiber membrane 3, and then a process for capturing and removing a virus at pores of the porous hollow fiber membrane 3 be repeatedly conducted.

The resin compound having an immobilized sugar chain according to the present invention may also be preferably used as a biocompatible material. There are many cases in which sugar chains present in the surface of cells, in general, and the resin compound having sugar chain according to the present invention exhibits high biocompatibility as a mimic material thereof. The biocompatible material according to the present invention may be used for medical purpose in, for example, a drug-delivery-system-material, a pH adjuster, a molding auxiliary material, a packaging material, an artificial blood vessel, a blood dialysis membrane, a catheter, a contact lens, a blood filter, a blood preservation pack, an artificial organ, or the like.

In the case where the resin compound having sugar chain according to the present invention is used as a biocompatible material, it may be preferably used as a material to form a film, molded product, or coating.

### EXAMPLES

The present invention will be explained further in detail with reference to the following examples.

### <Measurement of Pore Size of Porous Polymer Substrate>

The mean flow pore size (the mean pore size of recessed portions of pores extending from one side to the other side of a membrane) was measured in accordance with ASTM F316-86 and ASTM E1294-89 using a “Perm-Porometer CFP-200AEX” manufactured by Porous Materials, Inc., by a half-dry method. The test solution used was perfluoropolyester (under the trade name of “Galwick”).

### <Amount of Sugar Chain Immobilized in Resin Compound Having Immobilized Sugar Chain>

The amount of the sugar immobilized in a resin compound having an immobilized sugar chain was calculated from the dye adsorption amount of 1,9-dimethylmethylene blue.

Formation of calibration curve: A dye aqueous solution was prepared and mixed with a predetermined amount of the sugar to form a sugar-dye complex. The resultant was mixed with hexane to separate the sugar-dye complex from the aqueous phase, and then the amount of the dye remaining in the aqueous solution was determined by measuring the absorbance thereof (at 650 nm), to form a calibration curve using the amount of the sugar added and the absorbance.

Measurement of sample: A predetermined amount of a sample resin compound having an immobilized sugar chain was dissolved in a mixture of ethanol and water, and then the ethanol component was distilled away to obtain an aqueous dispersion of the resin compound having an immobilized sugar chain. 1,9-dimethylmethylene blue was added to the aqueous dispersion, and the dye adsorption amount was determined to calculate the amount of the immobilized sugar.

### <Calculation of Immobilized-Sugar Amount>

The amount of a sugar immobilized on a hollow fiber was calculated from the dye adsorption amount of 1,9-dimethylmethylene blue.

Formation of calibration curve: A dye aqueous solution was prepared and mixed with a predetermined amount of the sugar to form a sugar-dye complex. The resultant was mixed with hexane to separate the sugar-dye complex from the aqueous phase, and then the amount of the dye remaining in the aqueous solution was determined by measuring the absorbance thereof (at 650 nm), to form a calibration curve using the amount of the sugar added and the absorbance.

Measurement of sample: A hollow fiber with a predetermined length was put in a dye solution, and the dye adsorption amount was determined to calculate the amount of the immobilized sugar.

### <HCV Removal Test>

A hollow fiber module having a membrane area of 1.8 cm² was prepared, and 0.6 mL of the plasma (untreated fluid) collected from an HCV patient was passed through the module to obtain 0.3 mL of a fluid which had passed through pores thereof (filtrate) and 0.3 mL of a fluid which had not passed through the pores (internal solution). The sample was measured with an Ortho HCV antigen ELISA test, and the HCV removal ratio was calculated with the following formula.

HCV removal ratio(%)=(1−HCV load in filtrate/HCV load in untreated fluid)×100

### <ELISA Method>

The sample was pretreated with a pretreatment solution (SDS) so that the HCV core antigen was released and the HCV antibody present therewith was simultaneously deactivated to obtain a measurement sample. The measurement sample was put on an HCV core antigen-antibody-immobilized plate, and then incubated. After the reaction proceeded for a predetermined time, the resultant was rinsed, an HCV core antigen-antibody labeled with a horseradish peroxidase was added thereto, and then incubated. After the reaction proceeded for a predetermined time, the resultant was rinsed, an o-phenylenediamine reagent was added thereto, and then incubated. After the reaction proceeded for a predetermined time, a reaction-stop solution was added to the resultant. The color development was measured at a wavelength of 492 nm. The concentration was calculated using the absorbance of standard samples.

### <Calculation of Permeation Amount of Plasma Albumin>

A bromocresol green reagent was added to a sample, and the color development was measured at a wavelength of 630 nm. The concentration was calculated using the absorbance of standard samples.

Permeation ratio of albumin(%)=(amount of albumin in filtrate/amount of albumin in untreated fluid)×100

<Resin Solid Content (Mg)/Solvent Amount (Ml), at a Process for Obtaining a Hollow Fiber Having Immobilized Sugar Chain>

The resin solid content (mg) at the process for obtaining a hollow fiber having immobilized resin was determined by weight change of a hollow fiber between weights thereof measured before and after immobilization. On the other hand, the solvent amount (ml) at the process for obtaining the hollow fiber having immobilized resin was determined as a charge content of the solvent.

### Reference Example 1

### Preparation of Polymer Substrate

A high density polyethylene having a density of 0.968 g/cm³ and a melt index of 5.5 (HIZEX 2200J, manufactured by Mitsui Petrochemicals Industries, Ltd.) was spun with a hollow-fiber-forming spinneret having an extrusion orifice diameter of 16 mm, an annular slit width of 2.5 mm, and an extrusion cross section of 1.06 cm² at a spinning temperature of 160° C., and wound up at a spinning draft of 1890. The dimensions of the resultant undrawn hollow fiber were an inner diameter of 324 μm and a membrane thickness of 48 μm.

The undrawn hollow fiber was heated at 115° C. for 24 hours while being kept at a constant length. Subsequently, the fiber was subjected to drawing with a draw ratio of 1.8 at room temperature at a deformation rate of 7500%/min, then to hot drawing in a heating furnace at 100° C. at a deformation rate of 220%/min until total draw ratio reached 3.8, and further continuously to heat shrinkage in a heating furnace at 125° C. until total draw ratio reached 2.3, to obtain a drawn fiber. The resultant porous hollow fiber membrane had an inner diameter of 294 μm and a membrane thickness of 40 μm.

### Example 1

### Preparation of Epoxy Group-Introduced Ethylene-Vinyl Alcohol Copolymer (1)

170 parts by weight of ethylene-vinyl alcohol copolymer (manufactured by Nippon Synthetic Chemical Industry Co., Ltd., containing 44% by mole of ethylene, and having a weight-mean molecular weight of 90000), and 2380 parts by weight of dimethyl sulfoxide (manufactured by Wako Pure Chemical Industries., Ltd.) were placed in a four-necked flask equipped with a thermometer, a stirrer, a reflux condenser, and a nitrogen-gas inlet tube, and then heated to 90° C. to dissolve the ethylene-vinyl alcohol copolymer. Then, the temperature thereof was reduced to 50° C., and 2550 parts by weight of epichlorohydrin was added while conducting stirring to dissolve it. 85 parts by weight of 5% by weight of an aqueous sodium hydroxide solution was added thereto, and stirred the mixture while heating at 50° C. for 1 hour. Then, a resin component was precipitated using a reprecipitation technique, followed by conducting filtration, washing, and drying, to obtain an epoxy group-introduced ethylene-vinyl alcohol copolymer (1). The epoxy equivalent thereof was 2146 g/mol, and the weight-mean molecular weight thereof was 126000.

Preparation of Amino Group-Introduced Ethylene-Vinyl Alcohol Copolymer (1)

120 parts by weight of the epoxy group-introduced ethylene-vinyl alcohol copolymer (1), 1602 parts by weight of ethanol, and 678 parts by weight of ion-exchange water were placed in a four-necked flask equipped with a thermometer, a stirrer, a reflux condenser, and a nitrogen-gas inlet tube, followed by heating the mixture to 90° C. to dissolve the epoxy group-introduced ethylene-vinyl alcohol copolymer (1), and then reducing the temperature of the resultant to 40° C. The obtained solution of the epoxy group-introduced ethylene-vinyl alcohol copolymer (1) was added dropwise to a mixture solvent composed of 675 parts by weight of 28% by weight of ammonia water and 830 parts by weight of ethanol, followed by stirring the mixture at 40° C. for 4 hours. Then, 376 parts by weight of dimethyl sulfoxide was added to the resultant, and an excess ammonia component, ethanol, and water were distilled away to obtain a dimethyl sulfoxide solution of an amino group-introduced ethylene-vinyl alcohol copolymer (1) (in which an amine number of a solid content thereof was 25 mg KOH/g, and a non-volatile content was 5.9% by weight).

Preparation of Sugar Chain-Having Ethylene-Vinyl Alcohol Copolymer (1).

In a four-necked flask equipped with a thermometer, a stirrer, a reflux condenser, and a nitrogen-gas inlet tube, a mixture composed of 8.7 parts by weight of heparin (manufactured by LDO), 0.87 parts by weight of sodium cyanoborohydride, 44.6 parts by weight of ion-exchange water, and 103 parts by weight of dimethyl sulfoxide was added to 370 parts by weight of the dimethyl sulfoxide solution of the amino group-introduced ethylene-vinyl alcohol copolymer (1) (in which the non-volatile content was 5.9% by weight), and then the mixture was heated and stirred at 40° C. for 70 hours. Then, 32.8 parts by weight of acetyl chloride and 48.2 parts by weight of triethylamine were added to the resultant, and then reacted at 20° C. for 3 hours. Then, a resin component was precipitated using a reprecipitation technique, followed by conducting filtration, washing and drying, to obtain a sugar chain-having ethylene-vinyl alcohol copolymer (1). The amount of sugar contained in the resin compound having an immobilized sugar chain, measured using a dye adsorption technique, was 6.3% by weight.

### Example 2

The drawn fiber prepared in Reference Example 1 was immersed for 100 seconds in an immersion tank in which the sugar chain-having ethylene-vinyl alcohol copolymer (1) prepared in Example 1 was placed at 50° C., and kept warm under an ethanol saturated steam at 50° C. for 80 seconds, and then the hydrophilicity was provided to the resultant by drying the solvent for 80 seconds to obtain a hollow fiber having immobilized heparin. The amount of the immobilized heparin was determined by measuring a methylene blue adsorbing amount, and thereby it was revealed that the immobilized amount was 11 μg/cm² (calculated in terms of the inner surface area). The mean flow pore size of the hollow fiber was 137 nm. The ratio of resin solid content (mg)/solvent amount (ml), at the process for obtaining the hollow fiber in which the sugar chain-having ethylene-vinyl alcohol copolymer (1) was immobilized, was 40 mg/ml at a minimum.

### Example 3

A module was prepared using the hollow fiber prepared in Example 2, the plasma of an HCV patient was filtrated using the module, the amount of the HCV in the filtrate was measured using an ELISA method, and the adsorption and removal ratio (%) of the HCV was calculated. As a result, the adsorption ratio of the HCV was 52%. The permeation ratio of albumin was 99% or more.

### Example 4

### Evaluation of Biocompatibility

A slide glass was immersed for 10 minutes in a solution in which the sugar chain-having ethylene-vinyl alcohol copolymer (1) prepared in Example 1 was dissolved in a mixture solvent composed of ethanol and water at a concentration of 1% by weight. Then, the resultant was kept under an ethanol saturated steam at 50° C. for 80 seconds, and then further dried under an air atmosphere for 80 seconds to obtain a biocompatible material (1).

A protein solution having a protein concentration of 4 mg/mL was prepared by dissolving a BSA (bovine serum albumin), as a protein, in a 10 mM phosphate buffer having a pH of 7. The biocompatible material (1) was immersed at room temperature for 1.5 hours in the protein solution to attach the protein to the sample piece. Then, the resultant was washed at several times using purified water and dried, and then the absorbance of the biocompatible material (1) was measured at a wavelength of 560 nm using “UV-1650” manufactured by Shimadzu Corporation. The absorbance, relative to the absorbance of a substrate untreated with the protein, set as 100, was calculated as 30. The smaller the absorbance value was, the smaller the amount of the adsorbed protein was, and therefore the more superior the biocompatibility was.

### Comparative Example 1

The hollow fiber prepared in Reference Example 1 was treated using a 2.5% by weight ethanol/water mixture solution of ethylene-vinyl alcohol copolymer (manufactured by Nippon Synthetic Chemical Industry Co., Ltd., and having an ethylene content of 44% by mole and a weight-mean molecular weight of 90000) to provide the hydrophilicity to the resultant in the same way as that of Example 2. The thus obtained hollow fiber (about 13 cm, about 150 fibers: the amount of immobilized resin was 19.5 mg) was immersed in a test tube in which 20 mL of acetone, 16 mL of epichlorohydrin, and 4 mL of 40% by weight of an aqueous NaOH solution were placed. The reaction was caused while applying ultrasonic waves thereon at 30 to 40° C. for 5 hours, and, after the end of the reaction, the resultant was washed with acetone and water, and vacuum-dried to obtain an epoxy group-introduced hollow fiber.

The epoxy group-introduced hollow fiber was immersed in a 28% by weight ammonia water, and then reacted at 40° C. for 2 hours. After the end of the reaction, the resultant was washed with water to obtain a primary amino group-introduced hollow fiber. 40 mg of heparin and 4 mg of sodium cyanoborohydride were placed in a test tube, and dissolved with 40 mL of PBS, and then a hollow fiber was immersed therein to cause reaction at 40° C. for 1 day. After the end of the reaction, the resultant was washed with water. 26 mL of 0.2 M of an aqueous AcONa solution was placed on the resultant, and ice-cooled. 13 mL of an acetic anhydride was added dropwise at a slow speed while conducting ice-cooling. The reaction was caused by applying ultrasonic waves while conducting ice-cooling for 30 minutes. The reaction was further caused for 30 minutes while backing to room temperature. After the end of the reaction, the resultant was washed with 20% by weight of NaCl, 0.1 M of an aqueous NaHCO₃ solution, water, and PBS, to obtain a hollow fiber having immobilized heparin. The amount of immobilized heparin, determined by measuring a methylene blue adsorbing amount, was 10 μg/cm² (calculated in terms of the inner surface area). The mean flow pore size of the hollow fiber was 150 nm. The ratio of resin solid content (mg)/solvent amount (ml), at the process for obtaining the hollow fiber having immobilized heparin, was 0.5 mg/ml at a minimum.

### Comparative Example 2

A module was prepared using the hollow fiber prepared in Comparative Example 1, the plasma of an HCV patient was filtrated using the module, the amount of the HCV in the filtrate was measured using an ELISA method, and the adsorption and removal ratio (%) of the HCV was calculated. As a result, the adsorption ratio of the HCV was 49%. The permeation ratio of albumin was 99% or more.

### Comparative Example 3

The hollow fiber prepared in Reference Example 1 was treated using a 2.5% by weight ethanol/water mixture solution of ethylene-vinyl alcohol copolymer (manufactured by Nippon Synthetic Chemical Industry Co., Ltd., and having an ethylene content of 44% by mole and a weight-mean molecular weight of 90000) to provide the hydrophilicity to the resultant in the same way as that of Example 2. The mean flow pore size of the hollow fiber membrane was 139 nm. The hollow fiber was used to prepare a module, the plasma of an HCV patient was filtrated using the module, the amount of the HCV in the filtrate was measured using an ELISA method, and the adsorption and removal ratio (%) of the HCV was calculated. As a result, the adsorption ratio of the HCV was 29%. The permeation ratio of albumin was 99% or more.

### Comparative Example 4

An ethylene-vinyl alcohol copolymer (manufactured by Nippon Synthetic Chemical Industry Co., Ltd., and having an ethylene content of 44% by mole and a weight-mean molecular weight of 90000) was coated on the surface of a slide glass in the same way as that of Example 4 to obtain a comparative biocompatible material (1). A protein was adsorbed by the sample piece in the same way as that of Example 4, and then the absorbance thereof was measured, as a result of which was 105.

### INDUSTRIAL APPLICABILITY

The polymer substrate according to the present invention can be applied to a virus-removal apparatus, and the apparatus can be used to remove a virus.

The resin compound according to the present invention can be used as a biocompatible material for various medicinal purposes.

### DESCRIPTION OF THE REFERENCE SIGNS

1: virus fluid inflow port (first opening part)
2: outlet of fluid which has not passed through pores (second opening part)
3: porous hollow fiber membrane
4: outlet of fluid which has passed through pores (third opening part)
5: container
6: partition

1. A resin compound obtained by reacting a hydrophilic resin (A) selected from the group consisting of ethylene-vinyl alcohol copolymers, ethylene-acrylic acid copolymers, and ethylene-vinyl alcohol-vinyl acetate copolymers, with an epoxy-group-containing compound (B), followed by reacting an amino-group-containing compound (C) therewith, and then reacting a sugar with an amino group thereof, wherein the amino-group-containing compound (C) and the sugar are bonded by a covalent bond.

2. The resin compound according to claim 1, wherein the epoxy-group-containing compound (B) is an epichlorohydrin or a diepoxy compound.

3. The resin compound according to claim 1, wherein the amino-group-containing compound (C) is an ammonia, a methylamine, an ethylamine, a 2-aminoethanol, an ethylenediamine, a butylenediamine, a hexamethylenediamine, a 1,2-bis(2-aminoethoxy) ethane, a 3,3′-diaminodipropylamine, a diethylenetriamine, a phenylenediamine, a polyallylamine, or a polyethyleneimine.

4. The resin compound according to claim 1, wherein the sugar is a heparin, a heparin derivative obtained by subjecting a primary or secondary hydroxyl group of heparin to sulfuric-esterification, a heparin derivative obtained by removing an N-acetyl group from heparin to obtain a deacetylated heparin, and then subjecting the deacetylated heparin to N-sulfuric-esterification, a heparin derivative obtained by removing an N-sulfate group from heparin to obtain a desulfated heparin, and then subjecting the desulfated heparin to N-acetylation, a low-molecular-weight heparin, a dextran sulfate, a fucoidan, a chondroitin sulfate A, a chondroitin sulfate C, a dermatan sulfate, a heparinoid, a heparan sulfate, a rhamnan sulfate, a ketaran sulfate, an alginic acid, a hyaluronic acid, or a carboxymethyl cellulose.

5. The resin compound according to claim 1, wherein the hydrophilic resin (A) is an ethylene-vinyl alcohol copolymer or an ethylene-vinyl alcohol-vinyl acetate copolymer, in which a molar ratio of ethylene to vinyl alcohol, ethylene/vinyl alcohol, is within a range of 0.5 to 1.0.

6. A virus-removal-polymer substrate, comprising a surface coated with a resin compound of claim 1.

7. The virus-removal-polymer substrate according to claim 6, wherein a virus is a hepatitis virus.

8. The virus-removal-polymer substrate according to claim 6, wherein a polymer substrate is a porous hollow fiber, a non-woven fabric, or a dialysis membrane.

9. The virus-removal-polymer substrate according to claim 8, wherein the polymer substrate is a porous hollow fiber.

10. The virus-removal-polymer substrate according to claim 9, wherein the porous hollow fiber has a mean flow pore size within a range of 50 to 500 nm.

11. The virus-removal-polymer substrate according to claim 9, wherein the porous hollow fiber has an inner diameter within a range of 150 to 500 μm.

12. The virus-removal-polymer substrate according to claim 9, wherein the porous hollow fiber has a membrane thickness within a range of 30 to 100 μm.

13. A virus-removal-apparatus using a virus-removal-polymer substrate of claim 6.

14. A virus-removal-apparatus using a virus-removal-polymer substrate of claim 9.

15. A method for operating a virus-removal-apparatus of claim 14, comprising a step in which a fluid which has passed through pores of a porous hollow fiber and a fluid which has not passed through the pores thereof are mixed by passing a fluid comprising a virus through the porous hollow fiber.

16. The method for operating a virus-removal-apparatus according to claim 15, wherein the fluid comprising a virus is a blood comprising a virus.

17. A biocompatible material using a resin compound of claim 1.
